(19) United States
(12) Patent Application Publication
(10) Pub. No.: US 2017/0000650 A1
(43) Pub. Date: Jan. 5, 2017

(54) EAR COVER AND METHOD FOR MANUFACTURING THEREOF

(71) Applicant: Nodar Beneli, Gan Yavne (IL)

(72) Inventor: Nodar Beneli, Gan Yavne (IL)

(21) Appl. No.: 15/201,755

(22) Filed: Jul. 5, 2016

Related U.S. Application Data
(63) Continuation of application No. 13/710,492, filed on Dec. 11, 2012, now abandoned.

(51) Int. Cl. A61F 11/14; A61B 46/00; A61B 46/20

(52) U.S. Cl. CPC A61F 11/14; A61B 46/20; A61B 46/40; A61B 2046/205

(57) ABSTRACT

Disclosed is a protective sterile cover for the human ear. The cover may allow for the user to hear while protecting an ear that is sensitive to infection, and generally to protect an ear from water, fluids, hair and hair colorants, dust and other substances.

### FIELD OF THE INVENTION

The present invention relates to the field of protective covers for the human body. More particularly, the invention relates to an ear cover.

### BACKGROUND OF THE INVENTION

The ear, and especially the eardrum thereof, is very sensitive to infection, especially after ear surgery, upon entrance of water, other fluids and dust.

Also, rinsing the heads of babies may induce infections via accidental insertion of water into the ear. Carpentries, locksmith's workshop and other sites also are environments where non-desirable particles might enter the ear. Barbers and hairstylists also make efforts to prevent water, hair and dye entering the ear.

Ear corks are disadvantaged in that they block hearing. It is generally unaccepted to insert ear corks into children's ear, due to pain of not feeling the exact depth of insertion, and due to endangering perforation of the eardrum.

All the methods described above have not yet provided satisfactory solutions to the long felt need of protecting the ears while not reducing the hearing quality.

The problem is severe after ear surgery. Lack of a satisfactory solution to this problem where a patient has failed to take care, might cause complications of the surgical and medical care.

It is an object of the present invention to provide a device for protecting the ear while not reducing hearing quality.

It is an object of the present invention to provide a solution to the above-mentioned and other problems of the prior art.

Other objects and advantages of the invention will become apparent as the description proceeds.

### SUMMARY OF THE INVENTION

The present invention provides sterilization to the ear by covering the external ear, for preventing entrance of water and dust. This feature is very significant especially after ear surgery.

In one aspect, the present invention is directed to an ear cover (10), comprising:
a first sheet (12), the margin (16) thereof corresponding to an extent of an external ear (22), forming a pouch, for surrounding the helix (26) of the external ear (22); and
adhering means (14) disposed at a certain portion of the margin (16) of the first sheet (12), for sticking the margin portion to a front portion of the external ear (22), thereby sealing the cover (10) to the ear.

The first sheet (12) may be hollowed (32), for inserting medical tools therethrough, thereby the first sheet (12) provides sterilization to the ear by covering the external ear (22) during ear surgery.

At least a center portion (32) of the first sheet (12) may comprise a second sheet characterized to allow sound waves (54) to propagate therethrough with minimal reduction, thereby a user wearing the ear cover (10) is capable of hearing through the first sheet (12) substantially at the same level as without the ear cover (10).

Said at least center portion (32) of the first sheet (12) may comprise the entirety of the first sheet (12).

At least a center portion (32) of the first sheet (12) may comprise an optically transparent sheet, thereby providing esthetics as if the ear is not covered.

According to one embodiment, the second sheet (12) may comprise a thin and light sheet, thereby allowing sound waves (54) to propagate therethrough by minimally interrupting the propagation of the sound waves (54).

The second sheet may be loose.

The second sheet may comprise a material member selected from a group including: polyethylene, nylon.

According to another embodiment, the second sheet may comprise a sound membrane, thereby allowing sound waves (54) to propagate therethrough by receiving the sound waves (54) and transferring them forward to an eardrum of the ear.

The sound membrane sheet may be stretched by the first sheet (12), thereby providing the sound waves (54) receipt and the transfer thereof.

According to this embodiment, the sound membrane sheet may comprise a material member selected from a group including: a thin polymer, polycarbonate.

The first sheet (12) excluding the center (32) may comprise a relatively thick and flexible sheet, thereby providing improved mechanical characteristics for fastening the helix (26).

The first sheet (12) excluding the center (32) may comprise a material member selected from a group including: latex, silicon.

In another aspect, the present invention is directed to a method for manufacturing ear covers (10), the method comprising the steps of
providing a relatively large sheet (34);
placing a shaped knife on the sheet (34) for cutting thereof to a plurality of sheets (12), each shaped to cover one external ear (22);
attaching adhering means (14) to each cut sheet (12); and
attaching an elastic band to margins (16) of the cut sheets (12), for shrinking thereof.

The step of attaching the elastic band to the margins may comprise sewing.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof which are meant to be merely illustrative, and not limiting.

### BRIEF DESCRIPTION OF THE DRAWINGS

Embodiments and features of the present invention are described herein in conjunction with the following drawings:

FIG. 1 depicts an ear cover according to one embodiment of the present invention.

FIG. 2 depicts the ear cover of FIG. 1.

FIG. 3 is a side view of the ear cover of FIG. 1.

FIG. 4, shows the functions of the ear cover of FIG. 1.

FIG. 5 depicts steps of manufacturing ear covers of FIG. 1.

It should be understood that the drawings are not necessarily drawn to scale.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

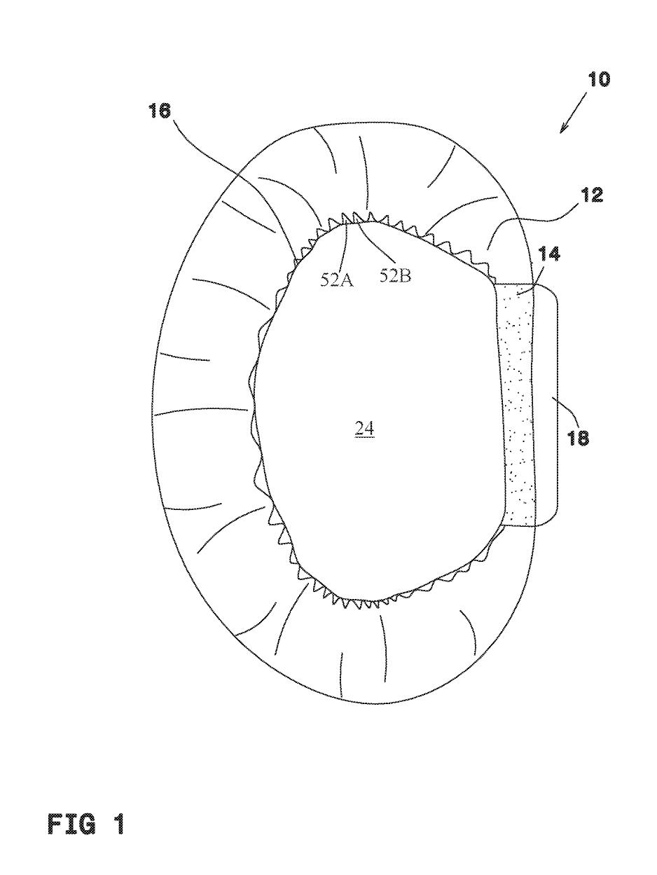

FIG. 1 depicts an ear cover according to one embodiment of the present invention.

The term “external ear” refers herein to the “pinna” which is the visible part of the ear residing outside of the head.

An ear cover 10 includes a sheet 12, that is shrunken to a form of a pouch having an opening 24 for inserting the external ear 22 therethrough. Sheet 12 is shrunken by an elastic band sewn or adhered to the margin 16 of sheet 12 by stitches 52A, 52B, etc.

Thus, margin 16 of sheet 12 surrounds the protruding rim 26 (helix) of external ear 22.

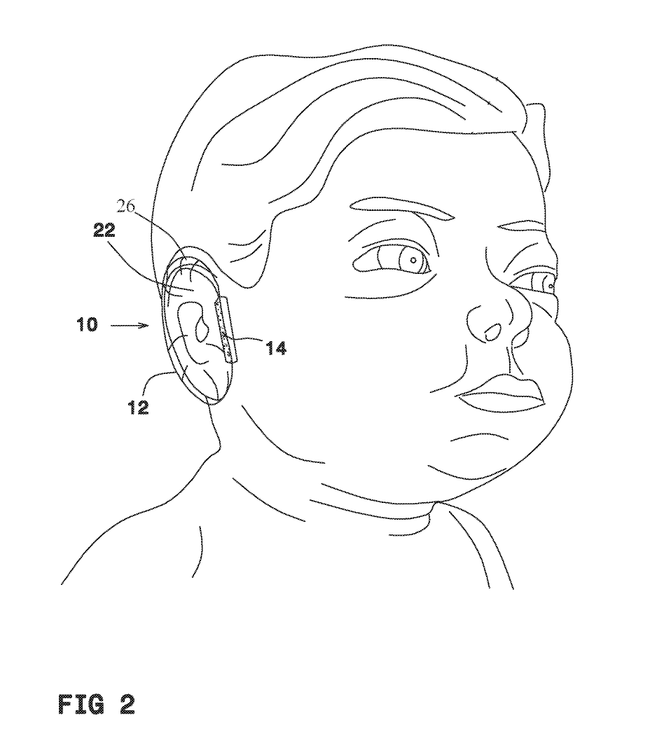

FIG. 2 depicts the ear cover of FIG. 1.

The elastic band is sufficient for attaching only the zones of external ear 12, having protrusions, which in FIGS. 1 and 2 are depicted up, left and down.

The zone of external ear 22 which does not protrude, and is shown at the right side, i.e., not belonging to helix 26, is adhered to sheet 12 by adhering means 14, these being a sticker. Sticker 14 is adhere-able to the front portion of external ear 22 upon separating a cover 18 therefrom.

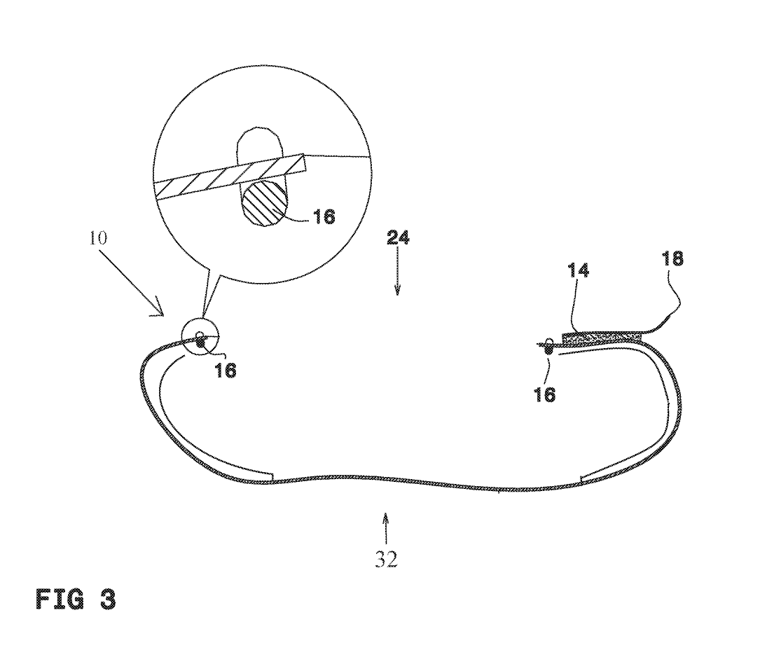

FIG. 3 is a side view of the ear cover of FIG. 1.

Opening 24 is depicted at the left. Sticker 14 and its 10 cover 18 are depicted on sheet 12 near opening 24. Margin 16 and the elastic band is depicted sewn inside and outside the page, for shrinking sheet 12, such that sheet 12 is not outspread.

According to a first embodiment, shrunken sheet 12 occupies the entire surface.

According to a second embodiment, the center 32 may constitute a different material than shrunken sheet 12.

According to a third embodiment, center 32 may be hollowed.

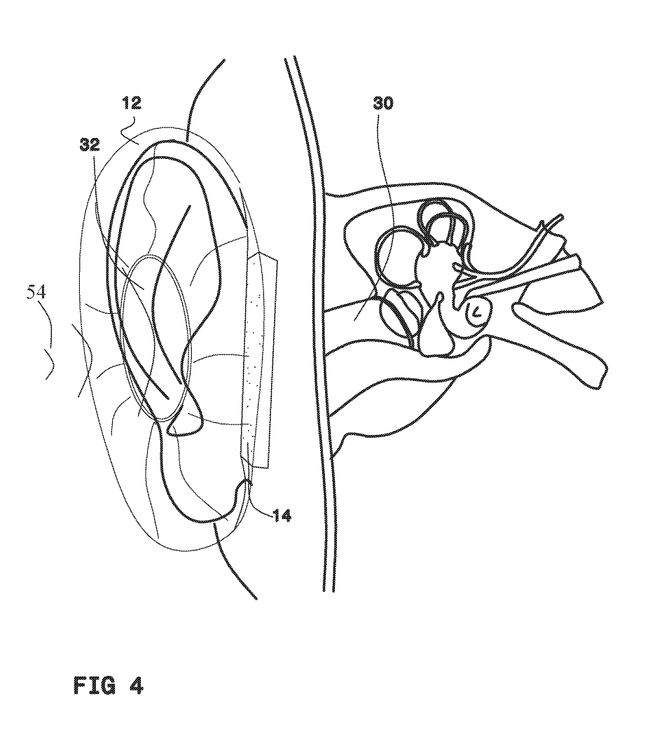

FIG. 4 shows the functions of the ear cover of FIG. 1.

According to all embodiments, center 32 is designed to allow sound waves 54 to propagate therethrough with minimal reduction, such that the wearer can clearly hear substantially at the same level as without wearing ear cover 10.

According to all embodiments, center 32 preferably is optically transparent, for viewing the ear, for providing esthetics as if the ear is not covered.

According to the third embodiment, center 32 is hollowed, and both the sound and the optical transparencies are obvious. This embodiment is intended for providing sterilization to external ear 22, while allowing a doctor to insert tools through hollowed center 32 into the internal ear 30 during ear surgery.

According to the first and second embodiments, in which center 32 is not hollowed, center 32 may constitute the embodiments of transferring sound waves 54 either by minimal interrupt, or via coherency.

The embodiment of transferring sound waves 54 by minimal interrupt consists of transferring sound waves 54 through a very thin and light sheet, such as a polyethylene or nylon sheet. According to this embodiment, the sheet at least at center 32 may be loose.

According to this embodiment, shrunken sheet 12, which fastens ear cover 10 to external ear 22, and the sheet of center 32, may both constitute the same single sheet.

According to the embodiment of transferring sound waves 54 via coherency, the sheet at center 32 functions as a sound drum/membrane, receiving sound waves 54 from the environment and transferring it forward to the eardrum. According to this embodiment, the sheet, at least at center 32, must be stretched and feature characteristics of a sound membrane. A thin and transparent polymer, such as polycarbonate, may be suitable for the sheet of center 32.

According to this embodiment, shrunken sheet 12, which fastens ear cover 10 to the external ear 22, and the sheet of center 32, preferably constitute different sheets, namely shrunken sheet 12 may be made of relatively thick latex or silicon, having preferred mechanical features for fastening external ear 22, and center 32 constitutes of the very thin polycarbonate being also optically transparent.

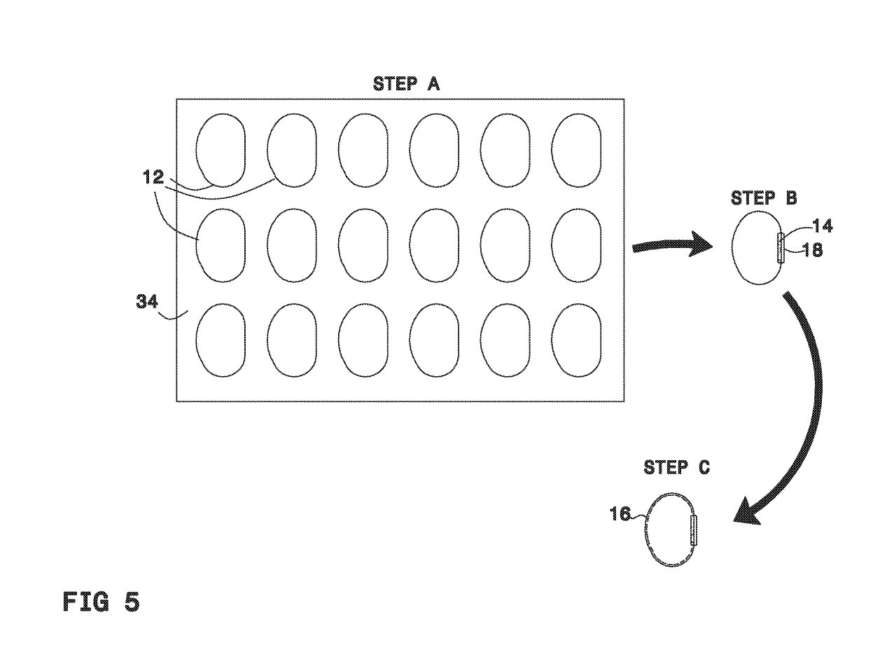

FIG. 5 depicts steps of manufacturing ear covers of FIG. 1.

The following description and the figure refers to the embodiment that shrunken sheet 12, which fastens ear cover 10 to external ear 22, and the sheet of center 32 may both constitute the same single sheet.

At step A, a large sheet 34 is cut to a plurality of small sheets 12, by placing a shaped knife thereon. Each small sheet 12 is sized to cover one external ear 22. The size may preferably be adapted to the appropriate ear size, depending whether it is for a baby, child or adult.

At step B, sticker 14 and its cover 18 are attached to each sheet 12.

At step C, an elastic band is sewn to each margin 16 of each sheet 12 by stitches 52A, 52B, etc.

In the figures and/or description herein, the following reference numerals have been mentioned:
numeral 10 denotes an ear cover according to one embodiment of the present invention;
numeral 12 denotes a shrunken sheet which fastens to the external ear; this shrunken sheet has an opening for wearing on the ear; this shrunken sheet may include a hole at the center or it may include another sheet at the center thereof;
numeral 14 denotes a sticker attached to the shrunken sheet, for adhering it to the front portion of the external ear;
numeral 16 denotes a margin of the sheet; an elastic band is sewn to this margin for shrinking the margin;
numeral 18 denotes a cover of the sticker;
numeral 22 denotes the external ear;
numeral 24 denotes an opening for wearing the ear cover;
numeral 26 denotes the helix of the external ear;
numeral 30 denotes the internal ear;
numeral 32 denotes a center portion of the shrunken sheet, which faces the external side of the external ear, for allowing sound to enter the inner ear; according to one embodiment, this center portion is hollowed; according to another embodiment, this center comprises a material allowing propagation of sound;
numeral 34 denotes a large sheet for producing a plurality of shrunken sheets therefrom;
numerals 52A, 52B, etc. denote stitches of the elastic band with the margin of the shrunken sheet; and
numeral 54 denotes a sound wave.

The foregoing description and illustrations of the embodiments of the invention has been presented for the purposes of illustration. It is not intended to be exhaustive or to limit the invention to the above description in any form.

Any term that has been defined above and used in the 15 claims, should to be interpreted according to this definition.

The reference numbers in the claims are not a part of the claims, but rather used for facilitating the reading thereof. These reference numbers should not be interpreted as limiting the claims in any form.

What is claimed is:

1. An ear cover (10), comprising:
a first sheet (12), the margin (16) thereof corresponding to an extent of an external ear (22), forming a pouch, for surrounding the helix (26) of said external ear (22); and
adhering means (14) disposed at a certain portion of said margin (16) of said first sheet (12), for sticking said margin portion to a front portion of said external ear (22).
thereby sealing said cover (10) to said ear.

2. An ear cover (10) according to claim 1, wherein said first sheet (12) is hollowed (32), for inserting medical tools therethrough,
thereby said first sheet (12) provides sterilization to said ear by covering said external ear (22) during ear surgery.

3. An ear cover (10) according to claim 1, wherein at least a center portion (32) of said first sheet (12) comprises a second sheet characterized to allow sound waves (54) to propagate therethrough with minimal reduction, thereby a user wearing said ear cover (10) is capable of hearing through said first sheet (12) substantially at the same level as without wearing said ear cover (10).

4. An ear cover (10) according to claim 3. wherein said at least center portion (32) of said first sheet (12) comprises the entirety of said first sheet (12).

5. An ear cover (10) according to claim 1, wherein at least a center portion (32) of said first sheet (12) comprises an optically transparent sheet, thereby providing esthetics as if the ear is not covered.

6. An ear cover (10) according to claim 3, wherein said second sheet (12) comprises a thin and light sheet, thereby allowing sound waves (54) to propagate therethrough by minimally interrupting said propagation of said sound waves (54).

7. An ear cover (10) according to claim 6, wherein said second sheet is loose.

8. An ear cover (10) according to claim 6, wherein said second sheet comprises a material member selected from a group including: polyethylene, nylon.

9. An ear cover (10) according to claim 3, wherein said second sheet comprises a sound membrane,
thereby allowing sound waves (54) to propagate therethrough by receiving said sound waves (54) and transferring them forward to an eardrum of the ear.

10. An ear cover (10) according to claim 9, wherein said sound membrane sheet is stretched by said first sheet (12),
thereby providing said sound waves (54) receipt and said transfer thereof.

11. An ear cover (10) according to claim 9, wherein said sound membrane sheet comprises a material member selected from a group including: a thin polymer, polycarbonate.

12. An ear cover (10) according to claim 9, wherein said first sheet (12) excluding said center (32) comprises a relatively thick and flexible sheet, thereby providing improved mechanical characteristics for fastening said helix (26).

13. An ear cover (10) according to claim 12, wherein said first sheet (12) excluding said center (32) comprises a material member selected from a group including: latex, silicon.

14. A method for manufacturing ear covers (10), said method comprising the steps of.
providing a relatively large sheet (34);
placing a shaped knife on said sheet (34) for cutting thereof to a plurality of sheets (12), each shaped to cover one external ear (22);
attaching adhering means (14) to each cut sheet (12); and
attaching an elastic band to margins (16) of said cut sheets (12), for shrinking thereof.

15. A method according to claim 14, wherein said step of attaching said elastic band to said margins comprises sewing.
